(19) United States
(12) Patent Application Publication
(10) Pub. No.: US 2023/0405047 A1
(43) Pub. Date: Dec. 21, 2023

(54) Methods and compositions for eliminating engineered immune cells

(71) Applicant: Miltenyi Biotec B.V. & Co. KG, Bergisch Gladbach (DE)

(72) Inventors: Dominik Lock, Bergisch Gladbach (DE); Caroline Brandes, Bergisch Gladbach (DE); Thomas Schaser, Bergisch Gladbach (DE)

(21) Appl. No.: 18/035,356

(22) PCT Filed: Nov. 5, 2021
(86) PCT No.: PCT/EP2021/080807

§ 371 Date: May 4, 2023

(30) Foreign Application Priority Data
Nov. 9, 2020 (EP) 20206353.3

(51) Int. Cl. A61K 35/17; C12N 5/078; C07K 14/045; A61K 39/00; C07K 14/725; A61P 37/06

(52) U.S. Cl. CPC A61K 35/17; C12N 5/0634; C07K 14/045; A61K 39/4631; C07K 14/7051; A61P 37/06; A61K 39/4621

(57) ABSTRACT

The present invention provides a composition comprising A) immune cells such as T cells comprising a) an inducible gene expression system comprising I) a first nucleic acid comprising a drug-inducible promoter operably linked to a second nucleic acid, and II) said second nucleic acid encoding a polypeptide or a non-coding RNA (ncRNA) which decreases cell surface expression level of major histocompatibility complex (MHC) class I relative to cell surface expression level of MHC class I of an immune cell that does not express said polypeptide or ncRNA; and b) a third nucleic acid encoding a chimeric antigen receptor (CAR) or T cell receptor (TCR); and B) a drug that induces said drug-inducible promoter. Preferentially, said polypeptide may be a viral protein which decreases cell surface expression level of major histocompatibility complex (MHC) class I relative to cell surface expression level of MHC class I of an immune cell that does not express the viral protein.

### FIELD OF THE INVENTION

The present invention generally relates to the field of immune cell immunotherapy, in particular to the drug-induced elimination of CAR T cells or T cells expressing a transgenic TCR in a subject.

### BACKGROUND OF THE INVENTION

The use of chimeric antigen receptor (CAR)-expressing T cells re-directed to specifically recognize and eliminate malignant cells, greatly increased the scope and potential of adoptive immunotherapy and is being assessed for new standard of care in certain human malignancies. CARs are recombinant receptors that typically target surface molecules in a human leukocyte antigen (HLA)-independent manner. Generally, CARs comprise an extracellular antigen recognition moiety, often a single-chain variable fragment (scFv) derived from antibodies or a Fab fragment, linked to an extracellular spacer, a transmembrane domain and intracellular co-stimulatory and signaling domains. Therapies using CAR-engineered T cells, although sometimes efficacious, have a high potential for improvements, especially with regard to safety [1, 2].

Immunoevasins belong to a heterogenous class of virus-derived proteins that interact with antigen presentation pathways by a direct interference with transcriptional regulation of genes or via post-translational modulation of antigen presentation and thereby allowing viruses to evade immune recognition [1].

WO2013/074916A1 discloses the use of zinc finger nucleases (or in alternative siRNA and TALENs) to disrupt T cell receptor α/β in CAR-expressing T cells and/or one or more human leukocyte antigen(s) (HLA) aiming to generate universal T cells that have lost both TCR- as well as HLA-expression less susceptible to immune-mediated recognition in an allogenic setting and thus improve persistency of CAR-engineered T cells.

In WO2018/132479 an isolated T lymphocyte with a) reduced or eliminated TCR expression and b) decreased HLA class I expression as well as c) expressing of a therapeutic protein is disclosed. Here TCR inactivation was carried out using e.g. ZFNs, TALENs or CRISPR/Cas9 system, HLA class I expression was modified using viral proteins including CMV US6, HSV TCP47, BoHV-1 UL49.5, and EBV BNLF2a. HLA class I-modified cells facilitate allogenic adoptive cellular therapy by evading rejection mediated by the immune system of a respective recipient.

WO2018/193394A1 discloses an isolated T cell comprising a) a viral protein which decreases surface expression of MHC class I and b) a chimeric antigen receptor to improve persistency of CAR-engineered T cells in allogenic setting. Here viral proteins were ICP47, K3, K5, E19, US3, US6, US2, U21, Nef, US10, or U21 derived from either CMV, adenovirus, herpesvirus, or human immunodeficiency virus (HIV).

WO2020/018691 discloses T lymphocytes with a) reduced or eliminated expression of TCR b) but expressing a heterologous viral protein facilitating evading immune response from a host to whom the T lymphocyte is administered and c) comprising a gene encoding for a therapeutic drug e.g. CAR. Viral proteins were derived from a virus of the family Herpesviridae, an adenovirus, an adeno-associated virus, an orthopoxviruses, or a retrovirus.

Aiming to develop an off-the-shelf CAR T cell product, as part of his PhD thesis, Benjamin Grimshaw (2015, University College London) investigated different strategies to reduce MHC I surface expression. Among others viral proteins including HSV-derived ICP47 and HMCV-derived US11 were used.

Existing safety mechanisms for CAR T cell immunotherapy such as suicide genes or extracellular tags that may be recognized by antibodies face problems in adoptive immune cell therapy [2, 3]. Thus, suicide genes may be immunogenic, have a low efficacy or function in actively proliferating cell or loss sensitivity upon a serial activation [2-4]. Recombinant tags may be also immunogenic and the elimination of the cells expressing the tag in a subject may incomplete [5].

There is a need in the art for an improved or alternative immune cell immunotherapy, e.g. a CAR T cell therapy, especially with regard to safety of this immunotherapy when applied to a subject.

### BRIEF DESCRIPTION OF THE INVENTION

The present inventors surprisingly found an effective cellular approach for controlled elimination of engineered immune cells such as CAR T cells. Autologous cells of a subject not presenting MHC class I on their cell surfaces anymore are recognized by autologous NK cells and eliminated (see FIG. 1). The controlled, i.e. drug-induced, reduction of MHC class I on the cell surface of an engineered immune cell such as a CAR T cell, surprisingly leads to a highly effective—and compared to existing safety mechanisms—a more extensive elimination of said engineered immune cells that have a reduced (decreased) MHC class I presentation on their cell surface. Here, the cellular mechanism of elimination is superior as compared to the elimination processes of engineered immune cells such as CAR T cells known in the art due to the lack of niches in the subject that may develop using said other engineered immune cell elimination processes.

Even more surprisingly it was found that the use of immunoevasins for induced reduction (decrease) of MHC class I on the cell surface is superior as compared to other mechanisms that lead to said reduction such as drug inducible CRISPR/Cas systems (or other gene editing tools) [6, 7], intrabodies or siRNA arranged in a manner that may knockout or knockdown the molecules of the MHC class I. It could be shown that immunoevasins are able to reduce (decrease) MHC class I on the cell surface of an immune cell such as a CAR T cell already within days or even within hours (depending on the kind of immunoevasin and its underlying mechanism) after they have been induced to be expressed in said cell. This may be due to the natural function of immunoevasins preventing the recognition of infected cells by immune cells of the host.

Further, the use if immunoevasins for reduction of MHC class I molecules on the surface of said immune cell expressing said CAR or transgenic TCR does not lead to off-target effects as observed with commonly known gene-editing tools like e.g. TALENs, ZFNs or CRISPR/Cas9.

The present invention provides a (pharmaceutical) composition comprising a) immune cells such as CAR T cells comprising a) an inducible gene expression system comprising a first nucleic acid comprising a drug-inducible promoter operably linked to a second nucleic acid, wherein the expression of said second nucleic acid decreases (reduces) cell surface expression level of major histocompatibility complex (MHC) class I relative to cell surface expression level of MHC class I of an immune cell such as a T cell that does not express said second nucleic acid, and b) a third nucleic acid encoding a chimeric antigen receptor (CAR) or a T cell receptor (TCR), and B) a drug that induces said drug-inducible promoter. Also provided are an in-vitro method for generating such engineered immune cells such as CAR T cells and a method for treatment of a subject suffering from a medical disease such as cancer with said composition. Furthermore, the present invention also provides a technical solution to improve the safety of a cellular product by an improved purity. Thus, during the manufacturing process unintentionally transduced immune cells such as e.g. B cells or leukemic B cells that might lead to a cancer relapse [8] would be eliminated if said second nucleic acid that decreases (reduces) cell surface expression level of major histocompatibility complex (MHC) class I relative to cell surface expression level of MHC class I of an immune cell such as a leukemic B cell that does not express said second nucleic acid, would be under the control of a B cell specific promoter.

Accordingly, said unintentionally transduced immune cell e.g. leukemic B cell would autonomously induce the expression of said second nucleic acid that decreases (reduces) cell surface expression level of major histocompatibility complex (MHC) class I.

### BRIEF DESCRIPTION OF THE DRAWINGS

FIG. 1: Schematic representation of a safety approach based on a reduced MHC I surface expression. Autologous NK cells eliminate T cells not expressing MHC I.

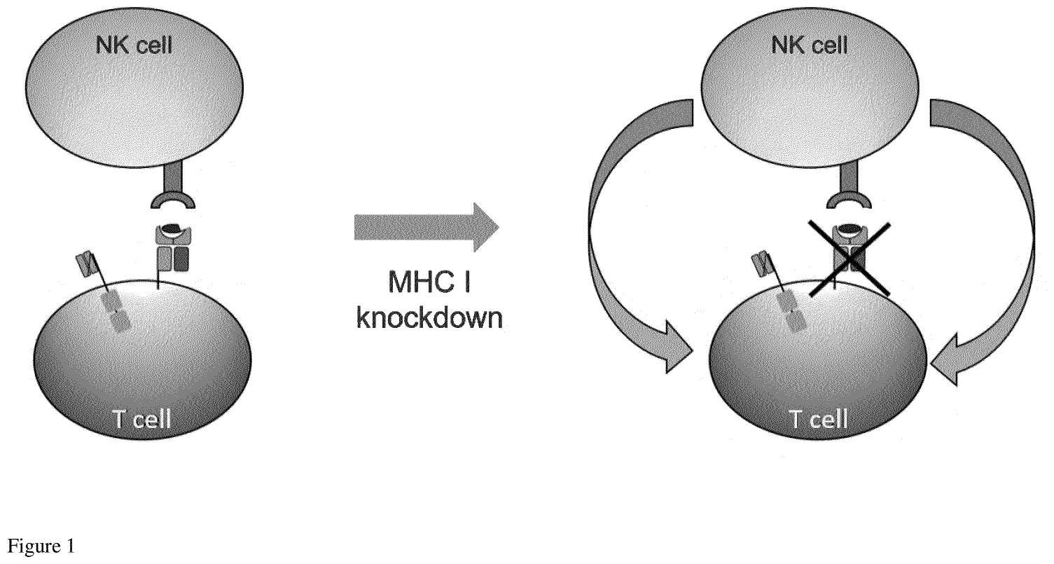

FIG. 2: T cells were transduced with lentiviral particles encoding the ICP47_P2A_eGFP safety construct. Transduction efficiency at day 6 was determined by the frequency of GFP positive (GFP+) cells using flow cytometry. Cells were stained with HLA-ABC-APC and analyzed using flow cytometry on d6 post transduction to assess ICP47-specific MHC-I downregulation. (A) Exemplifying dot plots and gating strategy of flow cytometry analysis. (B) Summary of flow cytometry analysis of 8 donors.

FIG. 3: (A) T cells were transduced with lentiviral particles encoding the ICP47_P2A_eGFP safety construct. Transduction efficiency at day 8 was determined by the frequency of GFP positive (GFP+) cells using flow cytometry. Cells were stained with HLA-ABC-APC and analyzed using flow cytometry on day 8 post transduction to assess ICP47-specific MHC-I downregulation. (B) ICP47-transduced T cells or Mock T cells were co-cultured with autologous NK cells using different E:T ratios (25:1, 5:1, 1:1, 0.2:1) for 18 h. Specific killing of T cells with reduced MHC I surface expression was determined using flow cytometry.

FIG. 4: T cells were transduced with lentiviral particles encoding either K3_P2A_eGFP, US6_P2A_eGFP or ICP47_P2A_eGFP safety construct, respectively. (A) Transduction efficiency at day 6 was determined by the frequency of GFP positive (GFP+) cells using flow cytometry. (B) Cells were stained with HLA-ABC-APC and analyzed using flow cytometry on d6 post transduction to assess immunoevasine-specific MHC-I downregulation. (C) K3-, US6-, ICP47-transduced T cells or Mock T cells were co-cultured with autologous NK cells using different E:T ratios (5:1, 1:1, 0.2:1) for 18 h. Specific killing of T cells with reduced MHC I surface expression was determined using flow cytometry.

### DETAILED DESCRIPTION OF THE INVENTION

In a first aspect, the present invention provides a composition comprising
A) immune cells such as T cells comprising
a) an inducible gene expression system comprising
I) a first nucleic acid comprising a drug-inducible promoter operably linked to a second nucleic acid
II) said second nucleic acid encoding a polypeptide (or protein) or a non-coding RNA (ncRNA) which (when expressed) decreases (reduces) cell surface expression level of major histocompatibility complex (MHC) class I relative to cell surface expression level of MHC class I of an immune cell such as a T cell that does not express said polypeptide or ncRNA
b) a third nucleic acid encoding a chimeric antigen receptor (CAR) or a T cell receptor (TCR),
B) a drug that induces said drug-inducible promoter.

Preferentially, said immune cells may be T cells.

Said immune cells may comprise T cells and B cells

Said immune cells may comprise T cells and leukemic B cells.

Leukemic B cells may be unintentionally transduced during the manufacturing process of the composition, i.e. the engineered immune cells such as T cells, and may be unintentionally applied to a subject suffering from leukemia and are therefore a severe safety risk for adoptive cellular therapies e.g. CAR T cell therapy. Thus, the administration of said drug (part B of said component) to said subject for eliminating said autologous engineered immune cells such as CAR T may also eliminate leukemic B cells, unintentionally transduced during the manufacturing process of said immune cells.

The composition as disclosed herein, wherein said second nucleic acid encoding a polypeptide (or protein) or a non-coding RNA (ncRNA) may be additionally operatively linked to a further nucleic acid (a fourth nucleic acid) comprising a tissue specific promoter.

Said tissue specific promoter may be a B cell specific promoter.

The composition as disclosed herein, wherein said second nucleic acid encoding a polypeptide (or protein) or a non-coding RNA (ncRNA) may be additionally operatively linked to a B cell specific promoter.

The composition as disclosed herein, wherein said second nucleic acid encoding a polypeptide (or protein) or a non-coding RNA (ncRNA) may be additionally operatively linked to a B cell specific promoter, wherein said B cell specific promoter may be transcriptionally more active in B cells than in non-B cells.

The composition as disclosed herein, wherein said second nucleic acid encoding a polypeptide (or protein) or a non-coding RNA (ncRNA) may be additionally operatively linked to a B cell specific promoter, wherein said polypeptide (or protein) or a non-coding RNA (ncRNA) may be expressed in B cells in the absence of said drug.

Said B cells may be healthy B cells and/or malignant B cells.

Said TCR may be a transgenic TCR.

Said second nucleic acid encoding a polypeptide (or protein) or a non-coding RNA (ncRNA) may be
i) a nucleic acid encoding a viral protein which (when expressed) decreases (reduces) cell surface expression level of major histocompatibility complex (MHC) class I relative to cell surface expression level of MHC class I of an immune cell (a T cell) that does not express the viral protein, or
ii) a nucleic acid encoding an antibody or antigen binding fragment thereof, that is intracellularly expressed, and that is specific for the alpha or beta2 microglobulin chain of MHC class I, or
iii) a ncRNA such as siRNA or miRNA specific for the alpha or beta2 microglobulin chain of MHC class I, or
iv) a nucleic acid encoding a polypeptide of a gene editing tool like for instance, but not limited to, ZFNs, TALENs CRISPR/Cas9 or MAD7 that are able to specifically knockout one of the following genes in said immune cell such a CAR T cell: alpha or beta2 microglobulin chains of MHC class I, or a protein of the transporter associated with antigen processing (TAP).

Preferentially, said second nucleic acid encoding a polypeptide (or protein) may a viral protein which (when expressed) decreases cell surface expression level of major histocompatibility complex (MHC) class I relative to cell surface expression level of MHC class I of an immune cell such as a T cell that does not express the viral protein.

Said viral protein which decreases cell surface expression level of major histocompatibility complex (MHC) class I relative to cell surface expression level of MHC class I of an immune cell such as a T cell that does not express the viral protein may be an immunoevasin.

Said CAR or said TCR may be expressed constitutively in said immune cell or said expression may be also inducible in said immune cell.

Said composition, wherein said cell surface expression level of MHC class I is reduced (decreased) on the cell surface, when said viral protein is expressed in said immune cell.

Said decrease/reduction of surface expression level of MHC class I may be at least 50%, at least 60%, at least 70%, at least 80%, at least 90%, at least 95%, or at least 99%. Said decrease may also be 100%.

Said decrease of cell surface expression level of MHC class I of at least at least 50%, at least 60%, at least 70%, at least 80%, at least 90%, at least 95%, or at least 99% may be reached, depending on the respective mechanism, after e.g. 12 hours (when MHC I gets actively degraded) or after 8 days (when MHC I turn-over is required) after induced expression of an immunoevasin in said immune cell.

Said viral protein may be from a virus selected from the group consisting of human cytomegalovirus (hCMV), murine cytomegalovirus (mCMV), rhesus cytomegalovirus (RhCMV), Epstein Barr virus (EBV), herpes simplex virus (HSV), bovine herpes virus-1 (BoHV-1), adenovirus (AV), coxpox virus (CV), Kaposi's sarcoma-associated herpesvirus (KSHV), mouse herpesvirus 68 (MHV68) or human immunodeficiency virus (HIV).

Said viral protein may be from hCMV and may be selected from the group consisting of US2, US3, US6, US10, US11, UL40, UL82, UL83, miR-3761, miR-US4-1 and UL18.

Said viral protein may inhibit transporter associated with antigen processing (TAP).

Said viral protein may be selected from the group consisting of US6, ICP47, UL49.5 and BNLF2a.

Said viral protein bay also be selected from the group consisting of EBNA1 (EBV-derived), E3-19K (AV-derived), CPXV203 (CV-derived), mK3 (MHV68-derived), gp48 (mCMV-derived), K3 (KSHV-derived), K5 (KSHV-derived) or Nef (HIV-derived).

The composition as disclosed herein, wherein said inducible gene expression system further may comprise a nucleic acid encoding a synthetic transcription factor for said drug-inducible promoter, wherein when a drug may be administered to said immune cell such as a T cell, the gene expression system may be induced (and the polypeptide (or protein) or ncRNA may be expressed).

Said synthetic transcription factor may comprise a DNA binding domain and drug-binding domain and an activation domain, wherein said synthetic transcription factor may be activated by binding to said drug.

Said drug may be a synthetic drug.

Said nucleic acid encoding said synthetic transcription factor may be operatively linked to a constitutive promoter.

Constitutive promoters may be for example EF-1 alpha promoter or any other constitutive promoter that drives constitutive expression in immune cells (such as MSCV, PGK-1, UBC, CMV, CAGG, SV40 or pan-hematopoietic promoter, such as vav).

Said synthetic transcription factor may e.g. comprise a DNA-binding protein or DNA-binding domain of a transcription factor (wildtype or engineered domain e.g. zinc finger protein or POU domain), a nuclear receptor and an activation domain, and wherein said drug may be a ligand of said nuclear receptor.

Said nuclear receptor may be e.g. the estrogen receptor (ER), the progesterone (PR)-, retinoid X- or the Drosophila ecdysone receptor.

Preferentially, said synthetic transcription factor may comprise a zinc finger protein, a nuclear receptor and an activation domain, and wherein said drug may be a ligand of said nuclear receptor.

Said synthetic transcription factor may comprise a zinc finger protein, the estrogen receptor (ER) and an activation domain, and wherein said drug may be tamoxifen or a tamoxifen metabolite.

Said activation domain may be e.g. herpes virus simplex protein VP16, the tetrameric repeat of VP16's minimal activation domain VP64, derived from the p65 domain of the human endogenous transcription factor NFxB or a fusion protein comprising sequence parts of the p65 domain of the human endogenous transcription factor NFxB and sequence parts of the human heat shock factor 1.

Said tamoxifen metabolite may be endoxifen or 4-hydroxytamoxifen (4-OHT).

Said ER may be an ER having point mutations such as murine ER (G525R or G521R), human ER (G400V, M543A, L540A) or human ER (G400V, M543A, L544A).

Said drug-inducible promoter may be a hybrid promoter comprising a zinc finger binding motif and a minimal promoter that comprises a minimal promoter e.g. selected from the group consisting of E1b, TK, IL2, CMV, SV40 or any minimal TATA box promoter.

Said composition as disclosed herein, wherein said synthetic transcription factor is a zinc finger protein.

Said composition as disclosed herein, wherein said synthetic transcription factor is a zinc finger protein, and wherein the level of expression of said polypeptide such as a viral protein or ncRNA depends on the amount of drug administered to said CAR T cell and/or on the number of binding sites (zinc finger binding motifs) for the DNA binding of the synthetic transcription factor within the drug-inducible promoter thereby allowing a tunable control of the expression of said polypeptide such as said viral protein or said ncRNA.

Said CAR may comprise
a) an antigen binding domain specific for an antigen
b) a transmembrane domain
c) an intracellular signaling domain,

Said antigen binding domain may be antibody or antigen binding fragment thereof such as a scFv or a Fab.

Alternatively said antigen binding domain may be ligand such as a cytokine that can bind to the cognitive receptor present on a target cell.

Said antigen may be an antigen expressed on the surface of a target cell such as a cancer cell.

Said antigen may be a soluble antigen, e.g. a soluble antigen that may be coupled to a solid surface or matrix such as a bead, or a soluble antigen that may allow for cross-linking, i.e. that induces dimerization of the CAR.

Said antigen may be a tagged polypeptide as disclosed herein. Then the antigen binding domain of said CAR may be specific for the tag, and the polypeptide may be bind to an antigen expressed on the surface of a target cell.

Said intracellular (cytoplasmic) signaling domain may comprise at least one primary cytoplasmic signaling domain comprising an immunoreceptor tyrosine-based activation motif (ITAM) and/or at least one co-stimulatory signaling domain.

Said primary cytoplasmic signaling domain of said first CAR may be CD3zeta.

Said at least one co-stimulatory domain of said CAR, may be selected from the group consisting of ICOS, CD154, CD5, CD2, CD46, HVEM, CD8, CD97, TNFRSF18, CD30, SLAM, DAP10, CD64, CD16, CD89, MyD88, KIR-2DS, KIR-3DS, NKp30, NKp44, NKp46, NKG2D, ICAM, CD27, OX40, 4-1BB, and CD28.

Said composition as disclosed herein, wherein said composition may comprise additionally, C) NK cells,

Wherein 90% of said immune cells such as T cells are eliminated within 24 hours by said NK cells, when said polypeptide (or protein) or ncRNA is expressed in said immune cell such as T cell and MHC I is downregulated on the surface of said immune cell such as T cell.

Said NK cells and said immune cells such as T cells may be autologous cells stemming from the same subject.

In a further aspect, the present invention provides a combination of components comprising a first component (A) and a second component (B), component A comprising immune cells such as T cells comprising
a) an inducible gene expression system comprising
I) a first nucleic acid comprising a drug-inducible promoter operably linked to a second nucleic acid
II) said second nucleic acid encoding a polypeptide (or protein) or ncRNA which (when expressed) decreases cell surface expression level of major histocompatibility complex (MHC) class I relative to cell surface expression level of MHC class I of an immune cell such as a T cell that does not express the polypeptide (or protein) or ncRNA
b) a third nucleic acid encoding a chimeric antigen receptor (CAR) or a TCR, and component B comprising a drug that induces said drug-inducible promoter.

In another aspect the present invention provides a composition (or a combination of components) for use in immunotherapy comprising
A) immune cells such as T cells comprising
a) an inducible gene expression system comprising
I) a first nucleic acid comprising a drug-inducible promoter operably linked to a second nucleic acid
II) said second nucleic acid encoding a polypeptide (or protein) or ncRNA which (when expressed) decreases cell surface expression level of major histocompatibility complex (MHC) class I relative to cell surface expression level of MHC class I of an immune cell such as a T cell that does not express the polypeptide (or protein) or ncRNA
b) a third nucleic acid encoding a chimeric antigen receptor (CAR) or a TCR,
B) a drug that induces said drug-inducible promoter.

Said immunotherapy may be for treatment of a medical condition such as autoimmune disease, cancer or infection.

Said composition for use in immunotherapy, wherein said immune cells such as T cells are autologous cells (of the subject to be treated).

Said immunotherapy may be for the treatment of cancer in a subject suffering from cancer.

Said immunotherapy may be for the treatment of cancer in a subject suffering from cancer, wherein said immune cells such as T cells are autologous cells of said subject.

Said composition for use in immunotherapy for reducing or preventing side-effects associated with an immunotherapy such as a CAR T cell therapy in a subject.

Said side-effects may be an on-target/off-tumor toxicity of engineered immune cells such as CAR T cells.

Said composition for use in immunotherapy for eliminating engineered immune cells such as CAR T cells in a subject.

In a further aspect the present invention provides a method for treatment of a subject suffering from a medical disease such as cancer, comprising
A) administration to said subject autologous immune cells such as T cells comprising
a) an inducible gene expression system comprising
I) a first nucleic acid comprising a drug-inducible promoter operably linked to a second nucleic acid
II) said second nucleic acid encoding a polypeptide (or protein) or ncRNA which (when expressed) decreases cell surface expression level of major histocompatibility complex (MHC) class I relative to cell surface expression level of MHC class I of an immune cell such as a T cell that does not express the polypeptide (or protein) or ncRNA
b) a third nucleic acid encoding a chimeric antigen receptor (CAR) or TCR, and
B) administration to said subject a drug that induces said drug-inducible promoter (thereby leading to the expression of said polypeptide (or protein) or ncRNA in said immune cell such as T cell).

Said method, wherein said inducible promoter in said immune cells such as T cells is capable of driving expression of said polypeptide (or protein) or ncRNA when said drug is administered to said immune cells such as T cells.

Said method, wherein said drug may be administered after the administration of said immune cells such as T cells to said subject.

In a further aspect the present invention provides a method for reduction or elimination of autologous engineered immune cells such as CAR T cells in a subject that has received said engineered immune cells such as CAR T cells for treatment of a medical disease such as cancer beforehand, wherein said engineered immune cells such as CAR T cells comprise
a) an inducible gene expression system comprising
I) a first nucleic acid comprising a drug-inducible promoter operably linked to a second nucleic acid
II) said second nucleic acid encoding a polypeptide (or protein) or ncRNA which (when expressed) decreases cell surface expression level of major histocompatibility complex (MHC) class I relative to cell surface expression level of MHC class I of an immune cell such as a T cell that does not express the polypeptide (or protein) or ncRNA
b) a third nucleic acid encoding a chimeric antigen receptor (CAR) or TCR,
the method comprising:
administration of a drug that induces said drug-inducible promoter (thereby leading to the expression of said polypeptide (or protein) or ncRNA in said immune cell such as T cell).

In another aspect the present invention provides an in-vitro method for generating engineered immune cells such as CAR T cells, the method comprising modifying immune cells such as T cells by introduction into said immune cells such as T cells
a) an inducible gene expression system comprising
I) a first nucleic acid comprising a drug-inducible promoter operably linked to a second nucleic acid
II) said second nucleic acid encoding a polypeptide (or protein) or ncRNA which (when expressed) decreases cell surface expression level of major histocompatibility complex (MHC) class I relative to cell surface expression level of MHC class I of an immune cell such as a T cell that does not express the polypeptide (or protein) or ncRNA
b) a third nucleic acid encoding a chimeric antigen receptor (CAR) or TCR,
wherein when a drug that induces said drug-inducible promoter is administered to said engineered immune cells such as CAR T cells, the gene expression system is induced and said polypeptide (or protein) or ncRNA is expressed.

Said immune cells such as T cells to be modified may be autologous immune cells such as T cells of a subject to be treated with said engineered immune cells such as CAR T cells.

Said immune cells such as T cells to be modified may be provided immune cells such as T cells from a subject to be treated with said engineered immune cells such as CAR T cells.

Said introduction may be a transduction using a retroviral vector such as a lentiviral vector.

Said in-vitro method, wherein said method is performed in a closed system.

Said in-vitro method, wherein said method is an automated method in a closed system.

Said in-vitro method, wherein said engineered immune cells such as CAR T cells are lysed by autologous NK cells, when said engineered immune cells such as CAR T cells are contacted with said autologous NK cells and when said drug is administered to said engineered immune cells such as CAR T cells.

In a further aspect the present invention provides a combination of pharmaceutical compositions comprising
A) a composition of immune cells such as T cells comprising
a) an inducible gene expression system comprising
I) a first nucleic acid comprising a drug-inducible promoter operably linked to a second nucleic acid
II) said second nucleic acid encoding a polypeptide (or protein) or ncRNA which (when expressed) decreases cell surface expression level of major histocompatibility complex (MHC) class I relative to cell surface expression level of MHC class I of an immune cell such as T cell that does not express the polypeptide (or protein) or ncRNA
b) a third nucleic acid encoding a chimeric antigen receptor (CAR) or TCR, and optional a pharmaceutical acceptable carrier, and
B) a composition of a drug that induces said drug-inducible promoter,
and optional pharmaceutical acceptable carrier.

Pharmaceutical acceptable carriers, diluents or excipients may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

All definitions, characteristics and embodiments defined herein with regard to the first aspect of the invention as disclosed herein also apply mutatis mutandis in the context of the other aspects of the invention as disclosed herein.

### Definitions

Unless defined otherwise, technical and scientific terms used herein have the same meaning as commonly understood by one of ordinary skill in the art to which this invention belongs. As used herein the term “comprising” or “comprises” is used in reference to compositions, methods, and respective component(s) thereof, that are essential to the method or composition, yet open to the inclusion of unspecified elements, whether essential or not.

The term “polypeptide (or protein) or ncRNA which decreases (or reduces) cell surface expression level of major histocompatibility complex (MHC) class I in an immune cell (a T cell) relative to cell surface expression level of MHC class I of an immune cell (a T cell) that does not express the polypeptide (or protein) or ncRNA” as used herein refer to heterologous polypeptides (or proteins) or ncRNAs that are able to reduce, down-regulate, knock down or knock out the expression of MHC class I in the cell, when they are expressed in said cell.

Preferentially, said polypeptide is a viral protein which decreases cell surface expression level of major histocompatibility complex (MHC) class I in an immune cell such as a T cell relative to cell surface expression level of MHC class I of an immune cell such as T cell that does not express the viral protein.

The term “viral protein which decreases cell surface expression level of major histocompatibility complex (MHC) class I in an immune cell such as a T cell relative to cell surface expression level of MHC class I of an immune cell such as a T cell that does not express the viral protein” as used herein refers to an immunoevasin that decreases (or reduces) the expression of MHC class I in the cell as compared to a cell that does not express said immunoevasin. The reduction (the decrease) of cell surface expression level of MHC class I may be an at least 50%, at least 60%, at least 70%, at least 80%, at least 90%, at least 95%, or at least 99% MHC class I reduction in said immune cell such as a T cell expressing said viral protein as compared to an immune cell such as a T cell that does not express said viral protein. Immunoevasins are proteins expressed by some viruses that enable the virus to evade immune recognition by preventing the presentation of viral peptides on MHC class I complexes on the infected cell. More specifically, an expression of an immunoevasin in a defined cell, e.g. in an immune cell such s a T cell will lead to a reduced major histocompatibility complex (MHC) class I expression in said cell.

The “immunoevasin” as used herein may also comprise a sequence having a sequence identity of at least 70%, or at least 75%, 80%, 85%, 90%, 95%, 97%, 98% or 99% at the amino acid sequence level to a natural (wild type) immunoevasins.

An immunoevasin may also be a functional fragment of a full-length immunoevasin or a fragment of a full length immunoevasin having a sequence identity of at least 70%, or at least 75%, 80%, 85%, 90%, 95%, 97%, 98% or 99% at the amino acid sequence level to said fragment of the natural immunoevasin. In this context “functional” means that the fragment is able to down-regulate the proteins of MHC class I in a sufficient manner (as done by the natural immunoevasin) as disclosed herein in a CAR T cell in that it is introduced.

Said (natural) immunoevasin may be a viral protein from a virus selected from the group consisting of human cytomegalovirus (hCMV), murine cytomegalovirus (mCMV), rhesus cytomegalovirus (RhCMV), Epstein Barr virus (EBV), herpes simplex virus (HSV), bovine herpes virus-1 (BoHV-1), adenovirus (AV), coxpox virus (CV), Kaposi's sarcoma-associated herpesvirus (KSHV), mouse herpesvirus 68 (MHV68) or human immunodeficiency virus (HIV).

For example, said viral protein may be from hCMV and may be selected from the group consisting of US2, US3, US6, US10, US11, UL40, UL82, UL83, miR-376a, miR-US4-1 and UL18.

Said viral protein may e.g. inhibit transporter associated with antigen processing (TAP), such viral proteins may be US6, ICP47, UL49.5 or BNLF2a.

Said viral protein bay also be selected from the group consisting of EBNA1 (EBV-derived), E3-19K (AV-derived), CPXV203 (CV-derived), mK3 (MHV68-derived), gp48 (mCMV-derived), K3 (KSHV-derived), K5 (KSHV-derived) or Nef (HIV-derived). Immunoevasins represent a heterogenous class of virus-derived proteins that interact with antigen presentation pathways by a direct interference with transcriptional regulation of genes or via post-translational modulation of antigen presentation and thereby allowing viruses to evade immune recognition [1].

HSV-derived ICP47 binds to the TAP1/2 dimer at the cytosolic face of the ER membrane and prevents peptide binding to TAP and transport into the lumen of the ER.

HCMV-derived US3 is a short-lived type I membrane glycoprotein that causes retention of certain MHC class I locus products in the ER [9].

HCMV-derived US6 protein binds to the core transmembrane domains of the TAP1/2 complex in the lumen of the ER and prevents peptide transport by inhibiting adenosine triphosphate (ATP) binding to the cytosolic adenosine triphosphatase (ATPase) cassettes of TAP1/2 [10].

The HCMV proteins US2 and US11 block class I MHC biosynthesis early in the secretory pathway by catalyzing the transport of newly synthesized, membrane-inserted class I MHC heavy chains to the cytosol where proteasomal degradation ensues [11].

The Kaposi's sarcoma-associated herpesvirus (KSHV)-derived immunoevasins K3 and K5, for instance, increase the endocytic rate of MHC I. Both act as E3 ubiquitin ligases that catalyze the ubiquitination of lysine residues in the cytosolic portions and thereby induce an active degradation of surface expressed MHC I [12]. In contrast to all mechanisms mentioned above, K3 and K5 are not dependent on MHC I turn-over kinetics (up to 8 days) but actively eliminate MHC I on the cell surface within hours [13].

A non-coding RNA (ncRNA) is an RNA molecule that is not translated into a protein.

As used herein “autologous” means that cells, a cell line, or population of cells used for treating subjects are originating from said subject.

As used herein “allogeneic” means that cells or population of cells used for treating subjects are not originating from said subject but from a donor.

Peptide fragments generated in the cytosol are displayed on class I major histocompatibility complex (MHC) products at the cell surface of antigen-presenting cells (APCs) or cells infected by a pathogen. The MHC is a large genetic complex with multiple loci. The MHC loci encode two major classes of MHC membrane molecules, referred to as class I and class II MHCs. T helper lymphocytes generally recognize antigen associated with MHC class II molecules, and T cytotoxic lymphocytes recognize antigen associated with MHC class I molecules. In humans the MHC is referred to as the HLA complex and in mice the H-2 complex.

MHC-I molecules are heterodimers, they have polymorphic heavy α-subunit whose gene occurs inside the MHC locus and small invariant 32 microglobulin subunit whose gene is located usually outside of it.

In general, a CAR as used herein may comprise an extracellular domain (extracellular part) comprising the antigen binding domain, a transmembrane domain and a cytoplasmic signaling domain (intracellular signaling domain). The extracellular domain may be linked to the transmembrane domain by a linker or spacer. The extracellular domain may also comprise a signal peptide. In some embodiments of the invention the antigen binding domain of a CAR binds a tag or hapten that is coupled to a polypeptide (“haptenylated” or “tagged” polypeptide), wherein the polypeptide may bind to a disease-associated antigen such as a tumor associated antigen (TAA) that may be expressed on the surface of a cancer cell. Such a CAR may be referred to as “anti-tag” CAR or “adapterCAR” or “universal CAR” as disclosed e.g. in U.S. Pat. No. 9,233,125B2.

The haptens or tags may be coupled directly or indirectly to a polypeptide (the tagged polypeptide), wherein the polypeptide may bind to said disease associated antigen expressed on the (cell) surface of a target. The tag may be e.g. dextran or a hapten such as biotin or fluorescein isothiocyanate (FITC) or phycoerythrin (PE) or thiamin, but the tag may also be a peptide sequence e.g. chemically or recombinantly coupled to the polypeptide part of the tagged polypeptide. The tag may also be streptavidin. The tag portion of the tagged polypeptide is only constrained by being a molecular that can be recognized and specifically bound by the antigen binding domain specific for the tag of the CAR. For example, when the tag is FITC (Fluorescein isothiocyanate), the tag-binding domain may constitute an anti-FITC scFv. Alternatively, when the tag is biotin or PE (phycoerythrin), the tag-binding domain may constitute an anti-biotin scFv or an anti-PE scFv, respectively.

A “signal peptide” refers to a peptide sequence that directs the transport and localization of the protein within a cell, e.g. to a certain cell organelle (such as the endoplasmic reticulum) and/or the cell surface.

Generally, an “antigen binding domain” refers to the region of the CAR that specifically binds to an antigen, e.g. to a tumor associated antigen (TAA) or tumor specific antigen (TSA). The CARs of the invention may comprise one or more antigen binding domains (e.g. a tandem CAR).

Generally, the targeting regions on the CAR are extracellular. The antigen binding domain may comprise an antibody or an antigen binding fragment thereof. The antigen binding domain may comprise, for example, full length heavy chain, Fab fragments, single chain Fv (scFv) fragments, divalent single chain antibodies or diabodies. Any molecule that binds specifically to a given antigen such as affibodies or ligand binding domains from naturally occurring receptors may be used as an antigen binding domain. Often the antigen binding domain is a scFv. Normally, in a scFv the variable regions of an immunoglobulin heavy chain and light chain are fused by a flexible linker to form a scFv. Such a linker may be for example the “(G₄/S)₃-linker”.

In some instances, it is beneficial for the antigen binding domain to be derived from the same species in which the CAR will be used in. For example, when it is planned to use it therapeutically in humans, it may be beneficial for the antigen binding domain of the CAR to comprise a human or humanized antibody or antigen binding fragment thereof. Human or humanized antibodies or antigen binding fragments thereof can be made by a variety of methods well known in the art.

“Spacer” or “hinge” as used herein refers to the hydrophilic region which is between the antigen binding domain and the transmembrane domain. The CARs of the invention may comprise an extracellular spacer domain but is it also possible to leave out such a spacer. The spacer may include e.g. Fc fragments of antibodies or fragments thereof, hinge regions of antibodies or fragments thereof, CH2 or CH3 regions of antibodies, accessory proteins, artificial spacer sequences or combinations thereof. A prominent example of a spacer is the CD8alpha hinge.

The transmembrane domain of the CAR may be derived from any desired natural or synthetic source for such domain. When the source is natural the domain may be derived from any membrane-bound or transmembrane protein. The transmembrane domain may be derived for example from CD8alpha or CD28. When the key signaling and antigen recognition modules (domains) are on two (or even more) polypeptides then the CAR may have two (or more) transmembrane domains. The splitting key signaling and antigen recognition modules enable for a small molecule-dependent, titratable and reversible control over CAR cell expression (e.g. WO2014127261A1) due to small molecule-dependent heterodimerizing domains in each polypeptide of the CAR.

The cytoplasmic signaling domain (the intracellular signaling domain or the activating endodomain) of the CAR is responsible for activation of at least one of the normal effector functions of the immune cell in which the CAR is expressed, if the respective CAR is an activating CAR (normally, a CAR as described herein refers to an activating CAR, otherwise it is indicated explicitly as an inhibitory CAR (iCAR)). “Effector function” means a specialized function of a cell, e.g. in a T cell an effector function may be cytolytic activity or helper activity including the secretion of cytokines. The intracellular signaling domain refers to the part of a protein which transduces the effector function signal and directs the cell expressing the CAR to perform a specialized function. The intracellular signaling domain may include any complete, mutated or truncated part of the intracellular signaling domain of a given protein sufficient to transduce a signal which initiates or blocks immune cell effector functions.

Prominent examples of intracellular signaling domains for use in the CARs include the cytoplasmic signaling sequences of the T cell receptor (TCR) and co-receptors that initiate signal transduction following antigen receptor engagement.

Generally, T cell activation can be mediated by two distinct classes of cytoplasmic signaling sequences, firstly those that initiate antigen-dependent primary activation through the TCR (primary cytoplasmic signaling sequences, primary cytoplasmic signaling domain) and secondly those that act in an antigen-independent manner to provide a secondary or co-stimulatory signal (secondary cytoplasmic signaling sequences, co-stimulatory signaling domain). Therefore, an intracellular signaling domain of a CAR may comprise one or more primary cytoplasmic signaling domains and/or one or more secondary cytoplasmic signaling domains.

Primary cytoplasmic signaling domains that act in a stimulatory manner may contain ITAMs (immunoreceptor tyrosine-based activation motifs).

Examples of ITAM containing primary cytoplasmic signaling domains often used in CARs are 15 that those derived from TCRζ (CD3ζ), FcRgamma, FcRbeta, CD3gamma, CD3delta, CD3epsilon, CD5, CD22, CD79a, CD79b, and CD66d. Most prominent is sequence derived from CD3ζ.

The cytoplasmic domain of the CAR may be designed to comprise the CD3ζ signaling domain by itself or combined with any other desired cytoplasmic domain(s). The cytoplasmic domain of the CAR can comprise a CD3ζ chain portion and a co-stimulatory signaling region (domain). The co-stimulatory signaling region refers to a part of the CAR comprising the intracellular domain of a co-stimulatory molecule. A co-stimulatory molecule is a cell surface molecule other than an antigen receptor or their ligands that is required for an efficient response of lymphocytes to an antigen. Examples for a co-stimulatory molecule are CD27, CD28, 4-1BB (CD137), OX40, CD30, CD40, PD-1, ICOS, lymphocyte function-associated antigen-1 (LFA-1), CD2, CD7, LIGHT, NKG2C, B7-H3.

The cytoplasmic signaling sequences within the cytoplasmic signaling part of the CAR may be linked to each other with or without a linker in a random or specified order. A short oligo- or polypeptide linker, which is preferably between 2 and 10 amino acids in length, may form the linkage. A prominent linker is the glycine-serine doublet.

As an example, the cytoplasmic domain may comprise the signaling domain of CD3ζ and the signaling domain of CD28. In another example the cytoplasmic domain may comprise the signaling domain of CD3ζ and the signaling domain of CD137. In a further example, the cytoplasmic domain may comprise the signaling domain of CD3ζ, the signaling domain of CD28, and the signaling domain of CD137.

As aforementioned either the extracellular part or the transmembrane domain or the cytoplasmic domain of a CAR may also comprise a heterodimerizing domain for the aim of splitting key signaling and antigen recognition modules of the CAR.

The CAR may be further modified to include on the level of the nucleic acid encoding the CAR one or more operative elements to eliminate CAR expressing immune cells by virtue of a suicide switch. The suicide switch can include, for example, an apoptosis inducing signaling cascade or a drug that induces cell death. In one embodiment, the nucleic acid expressing and encoding the CAR can be further modified to express an enzyme such thymidine kinase (TK) or cytosine deaminase (CD). The CAR may also be part of a gene expression system that allows controlled expression of the CAR in the immune cell. Such a gene expression system may be an inducible gene expression system and wherein when an induction agent is administered to a cell being transduced with said inducible gene expression system, the gene expression system is induced and said CAR is expressed on the surface of said transduced cell.

In some embodiments, the endodomain may contain a primary cytoplasmic signaling domains or a co-stimulatory region, but not both.

In some embodiment of the invention the CAR may be a “SUPRA” (split, universal, and programmable) CAR, where a “zipCAR” domain may link an intra-cellular costimulatory domain and an extracellular leucine zipper (WO2017/091546). This zipper may be targeted with a complementary zipper fused e.g. to an scFv region to render the SUPRA CAR T cell tumor specific. This approach would be particularly useful for generating universal CAR T cells for various tumors; adapter molecules could be designed for tumor specificity and would provide options for altering specificity post-adoptive transfer, key for situations of selection pressure and antigen escape.

If the CAR is an inhibitory CAR (referred to herein normally as “iCAR”) that may be expressed in addition to an activating CAR as described above in a cell, then said iCAR may have the same extracellular and/or transmembrane domains as the activating CAR but differs from the activating CAR with regard to the endodmain.

The at least one endodomain of the inhibitory CAR may be a cytoplasmic signaling domain comprising at least one signal transduction element that inhibits an immune cell or comprising at least one element that induces apoptosis.

Inhibitory endodomains of an iCAR are well-known in the art and have been described e.g. in WO2015075469A1, WO2015075470A1, WO2015142314A1, WO2016055551A1, WO2016097231A1, WO2016193696A1, WO2017058753A1, WO2017068361A1, WO2018061012A1, and WO2019162695A1.

The CARs of the present invention may be designed to comprise any portion or part of the above-mentioned domains as described herein in any order and/or combination resulting in a functional CAR, i.e. a CAR that mediated an immune effector response of the immune effector cell that expresses the CAR as disclosed herein.

The term “tagged polypeptide” as used herein refers to a polypeptide that has bound thereto directly or indirectly at least one additional component, i.e. the tag. The tagged polypeptide as used herein is able to bind an antigen expressed on a target cell. The polypeptide may be an antibody or antigen binding fragment thereof that binds to an antigen expressed on the surface of a target cell such as a tumor associated antigen on a cancer cell. The polypeptide of the tagged polypeptide alternatively may a cytokine or a growth factor or another soluble polypeptide that is capable of binding to an antigen of a target cell.

The terms “adapter” or “adapter molecule” or “tagged polypeptide” as used herein may be used interchangeably.

The tag may be e.g. a hapten or dextran and the hapten or dextran may be bound by the antigen binding domain of the polypeptide, e.g. a CAR, comprising an antigen binding domain specific for the tag.

Haptens such as e.g. FITC, biotin, PE, streptavidin or dextran are small molecules that elicit an immune response only when attached to a large carrier such as a protein; the carrier may be one that also does not elicit an immune response by itself. Once the body has generated antibodies to a hapten-carrier adduct, the small-molecule hapten may also be able to bind to the antibody, but it will usually not initiate an immune response; usually only the hapten-carrier adduct can do this.

But the tag may also be a peptide sequence e.g. chemically or recombinantly coupled to the polypeptide part of the tagged polypeptide. The peptide may be selected from the group consisting of c-Myc-tag, Strep-Tag, Flag-Tag, and Polyhistidine-tag. The tag may also be streptavidin. The tag portion of the tagged polypeptide is only constrained by being a molecular that can be recognized and specifically bound by the antigen binding domain specific for the tag of the CAR. For example, when the tag is FITC (Fluorescein isothiocyanate), the tag-binding domain may constitute an anti-FITC scFv. Alternatively, when the tag is biotin or PE (phycoerythrin), the tag-binding domain may constitute an anti-biotin scFv or an anti-PE scFv.

The term “antibody” as used herein is used in the broadest sense to cover the various forms of antibody structures including but not being limited to monoclonal and polyclonal antibodies (including full length antibodies), multispecific antibodies (e.g. bispecific antibodies), antibody fragments, i.e. antigen binding fragments of an antibody, immunoadhesins and antibody-immunoadhesin chimeras, that specifically recognize (i.e. bind) an antigen. “Antigen binding fragments” comprise a portion of a full-length antibody, preferably the variable domain thereof, or at least the antigen binding site thereof (“an antigen binding fragment of an antibody”).

Examples of antigen binding fragments include Fab (fragment antigen binding), scFv (single chain fragment variable), single domain antibodies (nanobodies), diabodies, dsFv, Fab′, diabodies, single-chain antibody molecules, and multispecific antibodies formed from antibody fragments. The antibody or antibody fragment may be human, fully human, humanized, human engineered, non-human, and/or chimeric. The non-human antibody or antibody fragment may be humanized to reduce immunogenicity to humans, while retaining the specificity and affinity of the parental non-human antibody. Chimeric antibodies may refer to antibodies created through the joining of two or more antibody genes which originally encoded for separate antibodies.

The terms “having specificity for”, “specifically binds” or “specific for” with respect to an antigen-binding domain of an antibody, of a fragment thereof or of a CAR refer to an antigen-binding domain which recognizes and binds to a specific antigen, but does not substantially recognize or bind other molecules in a sample. An antigen-binding domain that binds specifically to an antigen from one species may bind also to that antigen from another species. This cross-species reactivity is not contrary to the definition of that antigen-binding domain is specific. An antigen-binding domain that specifically binds to an antigen may bind also to different allelic forms of the antigen (allelic variants, splice variants, isoforms etc.). This cross reactivity is not contrary to the definition of that antigen-binding domain is specific.

As used herein, the term “antigen” is intended to include substances that bind to or evoke the production of one or more antibodies and may comprise, but is not limited to, proteins, peptides, polypeptides, oligopeptides, lipids, carbohydrates such as dextran, haptens and combinations thereof, for example a glycosylated protein or a glycolipid. The term “antigen” as used herein refers to a molecular entity that may be expressed e.g. on the surface of a target cell and that can be recognized by means of the adaptive immune system including but not restricted to antibodies or TCRs, or engineered molecules including but not restricted to endogenous or transgenic TCRs, CARs, scFvs or multimers thereof, Fab-fragments or multimers thereof, antibodies or multimers thereof, single chain antibodies or multimers thereof, or any other molecule that can execute binding to a structure with high affinity.

The term “soluble antigen” as used herein refers to an antigen that is not immobilized on surfaces such as beads or cell membranes.

The terms “immune cell” or “immune effector cell” may be used interchangeably and refer to a cell that may be part of the immune system and executes a particular effector function such as T cells, alpha-beta T cells, NK cells, NKT cells, B cells, innate lymphoid cells (ILC), cytokine induced killer (CIK) cells, lymphokine activated killer (LAK) cells, gamma-delta T cells, regulatory T cells (Treg), monocytes or macrophages. Preferentially these immune cells are human immune cells. Preferred immune cells are cells with cytotoxic effector function such as alpha-beta T cells, NK cells, NKT cells, ILC, CIK cells, LAK cells or gamma-delta T cells. Most preferred immune effector cells are T cells and NK cells. Tumor infiltrating lymphocytes (TILs) are T cells that have moved from the blood of a subject into a tumor. These TILs may be removed from a patient's tumor by methods well known in the art, e.g. enzymatic and mechanic tumor disruption followed by density centrifugation and/or cell marker specific enrichment. TILs are genetically engineered as disclosed herein, and then given back to the patient. “Effector function” means a specialized function of a cell, e.g. in a T cell an effector function may be cytolytic activity or helper activity including the secretion of cytokines.

T cells or T lymphocytes are a type of lymphocyte that play a central role in cell-mediated immunity. They can be distinguished from other lymphocytes, such as B cells and natural killer cells (NK cells), by the presence of a T cell receptor (TCR) on the cell surface. There are several subsets of T cells, each with a distinct function.

T helper cells (TH cells) assist other white blood cells in immunologic processes, including maturation of B cells into plasma cells and memory B cells, and activation of cytotoxic T cells and macrophages. These cells are also known as CD4+ T cells because they express the CD4 glycoprotein on their surface. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, including TH1, TH2, TH3, TH17, Th9, or TFH, which secrete different cytokines to facilitate a different type of immune response. Signaling from the APC directs T cells into particular subtypes.

Cytotoxic T cells (TC cells, or CTLs) destroy virally infected cells and tumor cells and are also implicated in transplant rejection. These cells are also known as CD8+ T cells since they express the CD8 glycoprotein at their surface. These cells recognize their targets by binding to antigen associated with MHC class I molecules, which are present on the surface of all nucleated cells.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus.

Two major classes of CD4+ Treg cells have been described—Foxp3+ Treg cells and Foxp3− Treg cells.

Natural killer T cells (NKT cells—not to be confused with natural killer cells of the innate immune system) bridge the adaptive immune system with the innate immune system. Unlike conventional T cells that recognize peptide antigens presented by major histocompatibility complex (MHC) molecules, NKT cells recognize glycolipid antigen presented by a molecule called CD1d. Once activated, these cells can perform functions ascribed to both Th and Tc cells (i.e., cytokine production and release of cytolytic/cell killing molecules).

The term “natural killer cells (NK cells)” are defined as large granular lymphocytes (LGL) and constitute the third kind of cells differentiated from the common lymphoid progenitor-generating B and T lymphocytes. NK cells are known to differentiate and mature in the bone marrow, lymph nodes, spleen, tonsils, and thymus, where they then enter the circulation. NK cells differ from natural killer T cells (NKTs) phenotypically, by origin and by respective effector functions; often, NKT cell activity promotes NK cell activity by secreting IFNγ. In contrast to NKT cells, NK cells do not express T-cell antigen receptors (TCR) or pan T marker CD3 or surface immunoglobulins (Ig) B cell receptors, but they usually express the surface markers CD16 (FcγRIII) and CD56 in humans, NK1.1 or NK1.2 in C57BL/6 mice. Up to 80% of human NK cells also express CD8. Continuously growing NK cell lines can be established from cancer patients and common NK cell lines are for instance NK-92, NKL and YTS.

Immunotherapy is a medical term defined as the “treatment of disease by inducing, enhancing, or suppressing an immune response”. Immunotherapies designed to elicit or amplify an immune response are classified as activation immunotherapies, while immunotherapies that reduce or suppress are classified as suppression immunotherapies. Cancer immunotherapy as an activating immunotherapy attempts to stimulate the immune system to reject and destroy tumors. Adoptive cell transfer uses cell-based, preferentially T cell-based or NK cell-based cytotoxic responses to attack cancer cells. T cells that have a natural or genetically engineered reactivity to a patient's cancer are generated in-vitro and then transferred back into the cancer patient. Then the immunotherapy is referred to as “CAR cell immunotherapy” or in case of use of T cells only as “CAR T cell therapy” or “CAR T cell immunotherapy”.

The term “treatment” as used herein means to reduce the frequency or severity of at least one sign or symptom of a disease.

The terms “therapeutically effective amount” or “therapeutically effective population” mean an amount of a cell population which provides a therapeutic benefit in a subject.

As used herein, the term “subject” refers to an animal. Preferentially, the subject is a mammal such as mouse, rat, cow, pig, goat, chicken dog, monkey or human. More preferentially, the subject is a human. The subject may be a subject suffering from a disease such as cancer (a patient) or from an autoimmune disease or from a allergic disease or from an infectious disease or from graft rejection.

The term “expression” as used herein is defined as the transcription and/or translation of a particular nucleotide sequence driven by its promoter in a cell.

The terms “engineered cell” and “genetically modified cell” as used herein can be used interchangeably. The terms mean containing and/or expressing a foreign gene or nucleic acid sequence which in turn modifies the genotype or phenotype of the cell or its progeny. Especially, the terms refer to the fact that cells, preferentially T cells can be manipulated by recombinant methods well known in the art to express stably or transiently peptides or proteins which are not expressed in these cells in the natural state. For example, T cells, preferentially human T cells are engineered to express an artificial construct such as a chimeric antigen receptor on their cell surface.

Genome editing, or genome engineering, or gene editing, is a type of genetic engineering in which DNA is inserted, disrupted, deleted, modified or replaced in the genome of a living organism. Unlike early genetic engineering techniques that randomly inserts genetic material into a host genome, genome editing targets the insertions to site specific locations. Prominent gene editing tools are Zinc finger nucleases (ZFNs), transcription-activator like effector nucleases (TALEN), meganucleases and the clustered regularly interspaced short palindromic repeats (CRISPR/Cas9) system.

The term “cancer” is known medically as a malignant neoplasm. Cancer is a broad group of diseases involving unregulated cell growth and includes all kinds of leukemia. In cancer, cells (cancerous cells) divide and grow uncontrollably, forming malignant tumors, and invading nearby parts of the body. The cancer may also spread to more distant parts of the body through the lymphatic system or bloodstream. There are over 200 different known cancers that affect humans.

The terms “nucleic acid”, “nucleic acid sequence/molecule” or “polynucleotide” as used interchangeably herein refer to polymers of nucleotides. Polynucleotides, which can be hydrolyzed into monomeric “nucleotides.” The monomeric nucleotides can be hydrolyzed into nucleosides. As used herein, the term “polynucleotides” encompasses, but is not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

As used herein, the terms “promoter” or “regulatory sequence” mean a nucleic acid sequence which is required for transcription of a gene product operably linked to the promoter/regulatory sequence. In some instances, this sequence may be the core promoter sequence and in other instances, this sequence may also include an enhancer sequence and other regulatory elements which are required for transcription of the gene product. The promoter/regulatory sequence may, for example, be one which expresses the gene product in a tissue-specific manner.

The term “minimal promoter (PMIN)” as used herein refers to the smallest genetic element that is able to induce transcription of a gene located downstream of said minimal promoter. Eukaryotic promoters of protein-coding genes have one or more of three conserved sequences in this region (i.e. the TATA-box, initiator region, and downstream promoter element). A minimal promoter enables low basal leakiness in the absence of specific transcriptional activators and high expression when transcription activators are bound upstream of minimal promoter at their specific DNA binding sites. Alternative minimal promoters can be used, such as minimal TATA box promoter, minimal CMV promoter or minimal IL-2 promoter.

The minimal promoter may be engineered/modified by the introduction of binding sites for specific transcription factors (e.g. required for the drug-inducible system).

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only in the presence or absence of certain conditions such as, for example, when an inducer (e.g. an induction signal, or an induction agent such as a drug, metal ions, alcohol, oxygen, etc.) is present in the cell.

Constitutive promoters that are operatively linked to a transgene may be for example EF-1 alpha promoter or any other constitutive promoter that drives constitutive expression in immune cells (such as MSCV, PGK-1, UBC, CMV, CAGG, SV40 or pan-hematopoietic promoter, such as vav).

In one variant the inducible promoter may be inducible by a drug, i.e. a drug-inducible promoter. The drug is selected based on safety record, favorable pharmacokinetic profile, tissue distribution, a low partition coefficient between the extracellular space and cytosol, low immunogenicity, low toxicities, and/or high expression in lymphocytes. In some alternatives, the inducible promoter is activated by a transcriptional activator (e.g. a synthetic transcription factor) that interacts with a drug. The transcriptional activator is activated or able to bind to and activate the inducible promoter in the presence of the drug. A specific alternative of a drug is a drug that binds to an estrogen receptor ligand binding domain of a transcriptional activator. In some alternatives, the drug includes tamoxifen, its metabolites, analogs, and pharmaceutically acceptable salts and/or hydrates or solvates thereof.

The term “synthetic transcription factor” as used herein may comprise a DNA-binding domain, a drug inducible domain (a drug binding domain) and an effector (activation) domain, that are linked and/or fused whereby the individual domains can be arranged in any order.

A DNA binding domain of a synthetic transcription factor may be a protein or a portion of a protein that specifically recognize the DNA binding motif of the drug-inducible promoter and mediate the binding of the synthetic transcription factor to this DNA sequence. Besides zinc finger proteins, TALE (transcription activator-like effector) and Cas9 (Clustered Regulatory Interspaced Short Palindromic Repeats-associated system) may be engineered to recognize a specific DNA sequence. Moreover, the DNA binding domain of naturally occurring transcription factors (e.g. POU homeodomain) may be employed.

Said DNA binding domain may be e.g. a zinc finger protein (or the DNA binding domain thereof) or a protein comprising or consisting of a POU domain.

DNA binding motifs of drug-inducible promoters are specific DNA sequences that are directly or indirectly (in case of Cas9) recognized by the DNA-binding domain of the synthetic transcription factor. E.g. each zinc finger domain specifically recognizes a DNA sequence of 3 bp, thus a three-finger zinc finger protein can be designed to recognize a 9 bp sequence.

Drug-binding domain of a synthetic transcription factor refers to a protein or a portion of a protein that binds to a drug or a ligand of the domain. Upon drug binding, the drug-binding domain enables the transition from an inactive to an active synthetic transcription factor. This transition may include the release of inactivation factors and/or the translocation of the synthetic transcription factor from the cytoplasm to the nucleus. Examples of drug binding domains are nuclear receptors, extracellular domains of receptors, antigen/substance binding proteins (also dimerizers) and/or active sites of enzymes.

An activation domain of a synthetic transcription factor refers to a protein or a portion of a protein that autonomously facilitates the recruitment of the transcriptional machinery to initiate mRNA transcription. Examples of activation domains are VP16, VP64, fragments of NFkB p65, heat shock factor 1 and combinations thereof.

E.g. the synthetic transcription factor may comprise a zinc finger protein, the estrogen receptor (ER) and an activation domain, and wherein said drug may be tamoxifen or a tamoxifen metabolite. Said activation domain may be e.g. herpes virus simplex protein VP16, the tetrameric repeat of VP16's minimal activation domain VP64, parts of the p65 domain of the human endogenous transcription factor NFxB or a fusion protein comprising fragments of human NFxB p65 and heat shock factor 1. Said tamoxifen metabolite may be endoxifen or 4-OHT. Said ER may be a ER having point mutations such as murine ER (G525R) or (G521R), human ER (G400V, M543A, L540A) or human ER (G400V, M543A, L544A).

The drug-inducible promoter may be a hybrid promoter comprising a DNA binding motif for said DNA binding domain of the synthetic transcription factor and a minimal promoter.

Said drug-inducible promoter may be a hybrid promoter comprising a zinc finger binding motif and a minimal promoter that comprises a minimal promoter selected from the group consisting of E1b, TK, IL2, CMV, SV40.

The term “inducible (gene) expression system” refers to the expression of an exogenous polypeptide (a transgene), herein normally the polypeptide such as a viral protein or ncRNA as disclosed herein in an immune cell.

The inducible gene expression system may be a drug-inducible gene expression system, i.e. the inducible gene expression system may be activated in a cell having said inducible gene expression system, when a drug, e.g. a synthetic drug such as tamoxifen may be introduced to the cell. Said drug in the cell may bind to a synthetic transcription factor and subsequently may lead to the induction of the expression of the transgene, herein normally the polypeptide such as the viral protein or the ncRNA as disclosed herein.

Said drug may also be referred to as “inducing agent”.

In the presence of an induction agent, the inducible expression system drives expression of the exogenous polypeptide. In an induced system, withdrawal of the induction agent may reduce and/or halt expression of the exogenous polypeptide. Upon re-introduction of the induction signal or the induction agent, the system can then be re-induced and restart the expression of the exogenous polypeptide, i.e. normally the polypeptide such as the viral protein or the ncRNA as disclosed herein.

In some embodiments, an inducible (gene) expression system as disclosed herein may also provide tunable control of the expression of the polypeptide such as the viral protein or the ncRNA. As used herein, the term “tunable control” refers to the ability to control the expression level of the polypeptide such as the viral protein or the ncRNA as disclosed herein. For example, the level of induced expression of the polypeptide such as the viral protein or the ncRNA as disclosed herein may depend on the amount of induction agent that is present. For example, the presence of a higher amount of induction agent, e.g. a synthetic drug may induce higher levels of expression of the polypeptide such as the viral protein or the ncRNA as compared to the presence of a lower amount of induction agent. As such, the inducible or tunable expression of the polypeptide such as the viral protein or the ncRNA may be dose-dependent with respect to the amount of induction agent present.

Besides the inducer drug dose, in some embodiments, an inducible (gene) expression system as disclosed herein may also provide tunable control of the expression of the polypeptide such as the viral protein or the ncRNA by the number of response elements for the synthetic transcription factor. As used herein, the term “tunable control” refers to the ability to control the expression level of the polypeptide such as the viral protein or the ncRNA as disclosed herein. For example, the level of induced expression of the polypeptide such as the viral protein or the ncRNA as disclosed herein may depend on the number of response elements in other words the number of binding sites for the synthetic transcription factor within the inducible promoter. For example, upon binding of five synthetic transcription factor molecules to an inducible promoter comprising five binding sites a transcriptional output i.e. a higher level of expression of the viral is induced as compared to constructs comprising two response elements within the inducible promoter. As such, the inducible or tunable expression of the polypeptide such as the viral protein or the ncRNA as disclosed herein may be dependent from the number of response elements for the synthetic transcription factor.

### EMBODIMENTS

In one embodiment of the invention the composition comprises
A) T cells comprising
a) an inducible gene expression system comprising
I) a first nucleic acid comprising a drug-inducible promoter operably linked to a second nucleic acid
II) said second nucleic acid encoding a viral protein which decreases cell surface expression level of major histocompatibility complex (MHC) class I relative to cell surface expression level of MHC class I of a T cell that does not express said viral protein
b) a third nucleic acid encoding a chimeric antigen receptor (CAR)
B) a drug that induces said drug-inducible promoter.

Said viral protein may be ICP47.

Said inducible gene expression system further may comprise a nucleic acid encoding a synthetic transcription factor for said drug-inducible promoter. Said nucleic acid encoding said synthetic transcription factor may be operatively linked to a constitutive promoter for example EF-1 alpha promoter.

Said drug-inducible promoter may be a hybrid promoter comprising a zinc finger binding motif and a minimal promoter.

Said synthetic transcription factor may comprise a zinc finger protein, the estrogen receptor (ER) and an activation domain, and wherein said drug may be tamoxifen or a tamoxifen metabolite. Said activation domain may be e.g. herpes virus simplex protein VP16.

Said tamoxifen metabolite may be endoxifen or 4-hydroxytamoxifen (4-OHT).

The CAR T cells of A) may be applied to a subject for treatment of a medical disease such as cancer, wherein said CAR T cells are autologous cell of said subject.

The CAR may comprise an antigen binding domain specific for a tumor associated antigen such as CD19. After treatment of the subject with said CAR T cells, the drug, e.g. 4-hydroxytamoxifen (4-OHT), may be applied to said subject for eliminating said autologous CAR T cells. The administration of said drug may lead to a reduction of cell surface expression level of MHC class I in said CAR T cells within about 8 days of at least 90%, thereby allowing autologous NK cells to recognize said CAR T cells and to eliminate them efficiently.

In another embodiment of the invention the viral protein may be Kaposi's sarcoma-associated herpesvirus (KSHV)-derived immunoevasins K3 and/or K5.

The drug and the drug-inducible promoter may be the same as disclosed in the first embodiment. Then, the administration of said drug leads to a reduction of cell surface expression level of MHC class I in said CAR T cells within 12 hours of at least 90%, thereby allowing autologous NK cells to recognize said CAR T cells and to eliminate them efficiently.

In another embodiment of the invention the viral protein may be selected from HCMV-derived immunoevasins including e.g. US2, US3, US6, US10, US11, UL40, UL82, UL83, miR-3761, miR-US4-1 and UL18.

The drug and the drug-inducible promoter may be the same as disclosed in the first embodiment. Then, the administration of said drug leads to a reduction of cell surface expression level of MHC class I in said CAR T cells within 8 days of at least 90%, thereby allowing autologous NK cells to recognize said CAR T cells and to eliminate them efficiently.

In another embodiment of the invention the viral protein may be selected from the group consisting of EBNA1 (EBV-derived), E3-19K (AV-derived), CPXV203 (CV-derived), mK3 (MHV68-derived), gp48 (mCMV-derived) or Nef (HIV-derived).

The drug and the drug-inducible promoter may be the same as disclosed in the first embodiment. Then, the administration of said drug leads to a reduction of cell surface expression level of MHC class I in said CAR T cells within 8 days of at least 90%, thereby allowing autologous NK cells to recognize said CAR T cells and to eliminate them efficiently.

In another embodiment of the invention diverse combinations of the viral proteins mentioned above (e.g., but not limited to, K3 and ICP47) may be used.

The drug and the drug-inducible promoter may be the same as disclosed in the first embodiment.

Then, the administration of said drug leads to a reduction of cell surface expression level of MHC class I in said CAR T cells within hours e.g. within 12 hours, when using e.g. K3 (or an equivalent immunoevasin having the same mechanism of action). Otherwise the administration of said drug leads to a reduction of cell surface expression level of MHC class I in said CAR T cells within 8 days of at least 90%. Autologous NK cells recognize said CAR T cells and eliminate them efficiently.

In another embodiment of the invention the composition comprises
A) T cells comprising
a) an inducible gene expression system comprising
I) a first nucleic acid comprising a drug-inducible promoter operably linked to a second nucleic acid
II) said second nucleic acid encoding a Transcription activator-like effector nuclease (TALEN) or a Zinc-finger nuclease (ZFN) designed to target a MHC class I molecule encoding gene for instance, but would not be limited to, the Beta2-microglobulin (B2M) encoding gene resulting in a respective knockout resulting in a disrupted MHC I surface expression relative to cell surface expression level of MHC class I of a T cell that does not express said B2M knockout.
b) a third nucleic acid encoding a chimeric antigen receptor (CAR),
B) a drug that induces said drug-inducible promoter.

The drug and the drug-inducible promoter may be the same as disclosed in the first embodiment. Then, the administration of said drug leads to the knockout of the MHC class I molecule such as B2M and the subsequent down-regulation of the cells surface expression level of MHC class I in said T cell.

In another embodiment of the invention the composition comprises
A) T cells comprising
a) an inducible gene expression system comprising
I) a first nucleic acid comprising a drug-inducible promoter operably linked to a second nucleic acid
II) said second nucleic acid encoding clustered, regularly interspaced, short palindromic repeats (CRISPR)-associated Cas9 protein
b) a third nucleic acid encoding a chimeric antigen receptor (CAR),
c) a RNA promoter driven single guide RNA (sgRNA) designed to target a MHC class I molecule encoding gene for instance, but would not be limited to, the Beta2-microglobulin (B2M) encoding gene resulting in a respective knockout resulting in a disrupted MHC I surface expression relative to cell surface expression level of MHC class I of a T cell that does not express said B2M knockout.
B) a drug that induces said drug-inducible promoter.

The drug and the drug-inducible promoter may be the same as disclosed in the first embodiment. Then, the administration of said drug leads to the knockout of the MHC class I molecule such as B2M and the subsequent down-regulation of the cells surface expression level of MHC class I in said T cell.

In another specific embodiment of the preceding embodiment of the invention the RNA promoter-driven sgRNA is under the control of an inducible system while the Cas9 can be constitutively expressed, for instance, but not limited to, c-terminally linked via a 2A element to the CAR or TCR encoding sequence.

In another embodiment of the invention a Cre induced transcription system could be used. Therefore, a loxP-stop-loxP (LSL) cassette can be placed in between a promoter and Cas9 coding sequence. Induction of Cre would lead to a Cre-mediated loxP recombination which removes the stop signal, thus activating Cas9 expression.

In another embodiment of the invention the composition comprises
A) a leukemic B cell comprising, e.g. as part of a T cell composition obtained from a subject a) an inducible gene expression system comprising
I) a first nucleic acid comprising a drug-inducible promoter operably linked to a second nucleic acid
II) said second nucleic acid encoding a viral protein which decreases cell surface expression level of major histocompatibility complex (MHC) class I relative to cell surface expression level of MHC class I of a leukemic B cell that does not express said viral protein
b) a third nucleic acid encoding a chimeric antigen receptor (CAR),
B) a drug that induces said drug-inducible promoter.

Said viral protein may be, but not limited to, ICP47, K3, K5, US2, US3, US6 or US11 as well as combinations thereof.

Said inducible gene expression system further may comprise a nucleic acid encoding a synthetic transcription factor for said drug-inducible promoter. Said nucleic acid encoding said synthetic transcription factor may be operatively linked to a constitutive promoter for example EF-1 alpha promoter.

Said drug-inducible promoter may be a hybrid promoter comprising a zinc finger binding motif and a minimal promoter.

Said synthetic transcription factor may comprise a zinc finger protein, the estrogen receptor (ER) and an activation domain, and wherein said drug may be tamoxifen or a tamoxifen metabolite. Said activation domain may be e.g. herpes virus simplex protein VP16.

Said tamoxifen metabolite may be endoxifen or 4-hydroxytamoxifen (4-OHT).

Engineered leukemic B cells of A) may be unintentionally applied to a subject and are therefore a severe safety risk for adoptive cellular therapies e.g. CAR T cell therapy. In this context the CAR expressed on T cells may comprise an antigen binding domain specific for a tumor associated antigen such as CD19. Engineered leukemic B cells, however, might cause a progressive leukemia. Thus, the drug, e.g. 4-hydroxytamoxifen (4-OHT), may be applied to said subject for eliminating not only said autologous CAR T cells but also leukemic B cells, unintentionally transduced during the manufacturing process contaminating the cellular product. The drug and the drug-inducible promoter may be the same as disclosed in the first embodiment. Then, the administration of said drug leads to a reduction of cell surface expression level of MHC class I in said leukemic B cells dependent on the used immunoevasin within 12 hours (if K3 or K5 or another immunoevasins of this class is used) or within 8 days of at least 90%, thereby allowing autologous NK cells to recognize said engineered leukemic B cells and to eliminate them efficiently.

In another embodiment of this invention a viral protein as mentioned above is driven under a B cell specific promoter leading to a constitutive expression of said viral protein in B cells, healthy as well as malignant. Thus, unintentionally transduced B cells, e.g. a leukemic B cell, will directly express the viral protein which will lead to a reduction of cell surface expression level of MHC class I in said B cell dependent on the used immunoevasin within 12 hours (if K3 or K5 or another immunoevasin of this class is used) or within 8 days of at least 90%, thereby allowing autologous NK cells to recognize said B cells and to eliminate them efficiently.

In another embodiment of this invention Transcription activator-like effector nuclease (TALEN), Zinc-finger nuclease (ZFN) or clustered, regularly interspaced, short palindromic repeats (CRISPR)/Cas9 designed to target a MHC class I molecule for instance, but would not be limited to, the Beta2-microglobulin (B2M) encoding gene might be used resulting in a respective knockout resulting in a disrupted MHC I surface expression relative to cell surface expression level of MHC class I of a unintentionally genetically engineered leukemic B cell that does not express said B2M knockout might be used as described in previous embodiments.

In another embodiment of the invention the composition comprises
A) T cells comprising
a) an inducible gene expression system comprising
I) a first nucleic acid comprising a drug-inducible promoter operably linked to a second nucleic acid
II) said second nucleic acid encoding a viral protein which decreases cell surface expression level of major histocompatibility complex (MHC) class I relative to cell surface expression level of MHC class I of a T cell that does not express said viral protein
b) a third nucleic acid encoding a transgenic T cell receptor (TCR),
B) a drug that induces said drug-inducible promoter.

Said viral protein may be, but not limited to, ICP47, K3, K5, US2, US3, US6 or US11 as well as combinations thereof.

Said inducible gene expression system further may comprise a nucleic acid encoding a synthetic transcription factor for said drug-inducible promoter. Said nucleic acid encoding said synthetic transcription factor may be operatively linked to a constitutive promoter for example EF-1 alpha promoter.

Said drug-inducible promoter may be a hybrid promoter comprising a zinc finger binding motif and a minimal promoter.

Said synthetic transcription factor may comprise a zinc finger protein, the estrogen receptor (ER) and an activation domain, and wherein said drug may be tamoxifen or a tamoxifen metabolite. Said activation domain may be e.g. herpes virus simplex protein VP16.

Said tamoxifen metabolite may be endoxifen or 4-hydroxytamoxifen (4-OHT).

After treatment of the subject with said TCR engineered T cells, the drug, e.g. 4-hydroxytamoxifen (4-OHT), may be applied to said subject for eliminating said autologous TCR engineered T cells. The drug and the drug-inducible promoter may be the same as disclosed in the first embodiment. Then, the administration of said drug leads to a reduction of cell surface expression level of MHC class I in said TCR engineered T cells dependent on the used immunoevasin within 12 hours (if K3 or K5 or another immunoevasin of this class is used) or within 8 days of at least 90%, thereby allowing autologous NK cells to recognize said TCR engineered T cells and to eliminate them efficiently.

In another embodiment of this invention Transcription activator-like effector nuclease (TALEN), Zinc-finger nuclease (ZFN) or clustered, regularly interspaced, short palindromic repeats (CRISPR)/Cas9 designed to target a MHC class I molecule for instance, but would not be limited to, the Beta2-microglobulin (B2M) encoding gene might be used resulting in a respective knockout resulting in a disrupted MHC I surface expression relative to cell surface expression level of MHC class I of a unintentionally TCR engineered T cells that does not express said B2M knockout might be used as described in previous embodiments.

### EXAMPLES

### Example 1. Genetic Modification of T Cells with ICP47-Encoding Lentiviral Particles

### 1.1 Construct Design and Production of Lentiviral Particles

In order to develop a constitutively expressing ICP47 lentiviral vector, the ICP47 encoding sequence (UniProt ID: P03170) was subcloned under the control of a PGK promoter. To visualize the transduction a GFP encoding sequence was c-terminally linked using a 2A element. Vesicular stomatitis virus glycoprotein (VSV-G)-pseudotyped lentiviral vectors were produced using HEK 293-T cells. 1.6E7 cells were seeded in 20 ml medium in a T175 flask 20 h prior to transfection. 3.15 μg VSV-G encoding plasmid pMDG-2, 19.37 μg gag/pol/rev encoding plasmid pCMVdR8.74 and 12.59 pg transfer vector plasmid were diluted in 3.5 ml DMEM without additives and mixed with 3.5 ml DMEM supplemented with 280 μl PEI (1 mg/ml). The transfection mixture was incubated for 20 min at RT. HEK 293-T medium was completely removed from the cells and replaced with 16 ml DMEM without additives. Subsequently, the transfection mixture was carefully added to the cells. After 4-6 h 2.5 ml FCS were added and cultured for 24 h before 520 μl sodium butyrate (500 mM) was additionally added. 48 h after transfection, supernatant was collected, sterile filtrated and concentrated at 4° C. for 24 h at 5350 g. Pelleted lentiviral particles were diluted in PBS and freshly used or stored at −70° C.

### 1.2 Isolation, Expansion and Modification of T Cells

Unless mentioned to the contrary, kits and reagents were used according to the manufacturer's protocol. All kits and reagents, unless mentioned otherwise, were from Miltenyi Biotec.

T cells were either isolated from freshly isolated or frozen PBMC using the human PAN T cell isolation Kit or derived from whole blood using a CD3 depletion fraction after staining with CD3-Microbeads and depletion using LD-columns. Autologous NK cells were either directly isolated from the same donor using the NK cell isolation Kit or isolated from whole blood after CD3 depletion and enrichment of CD56+ cells via CD56-Microbeads.

T cells were activated in TexMACS supplemented with 12.5 ng/ml recombinant human IL-7 and 12.5 ng/ml recombinant human IL-15 as well as MACS GMP T Cell TransAct with a titer of 1:17.5 or T cell TransAct, human with a titer of 1:100. For this purpose, 1E6 T cells per cm² were cultured in 1 ml medium for 72 h at 37° C. and 5% C02 atmosphere before the stimulation reagent was removed. From then onwards T cells were cultured in 24-well plates in 2 ml TexMACS supplemented with 12.5 ng/ml recombinant human IL-7 and 12.5 ng/ml recombinant human IL-15. T cells were splitted 1:2 every other day.

1E6 NK cells per cm² were cultured in 1 ml NK MACS Medium supplemented with 1% NK MACS Supplement, 5% heat inactivated AB serum, 500 IU/ml IL2, 140 IU/ml IL15 as well as, for the first three days, 80 ng/ml IL-1beta.

T cells were transduced 24 h after activation with fresh or frozen, carefully resuspended VSV-G pseudotyped lentiviral particles.

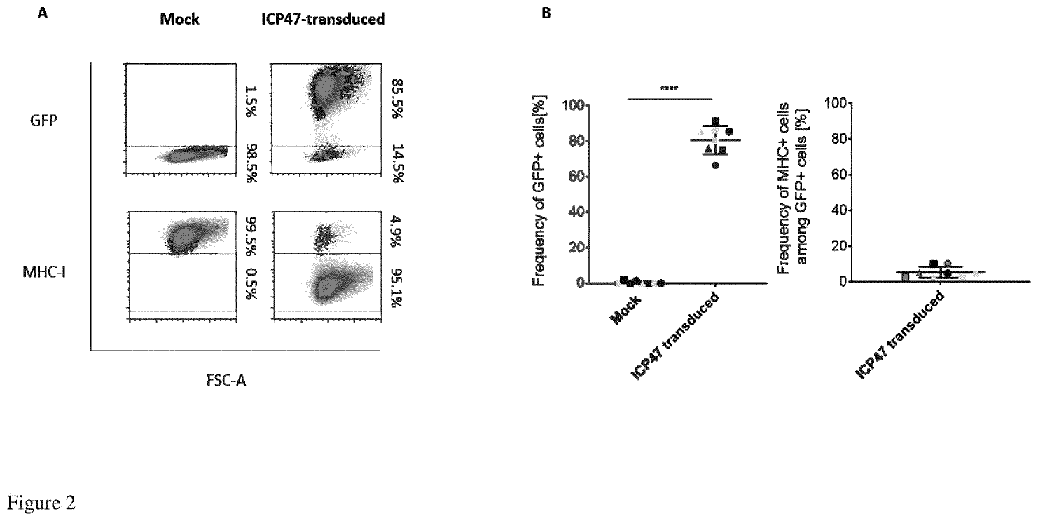

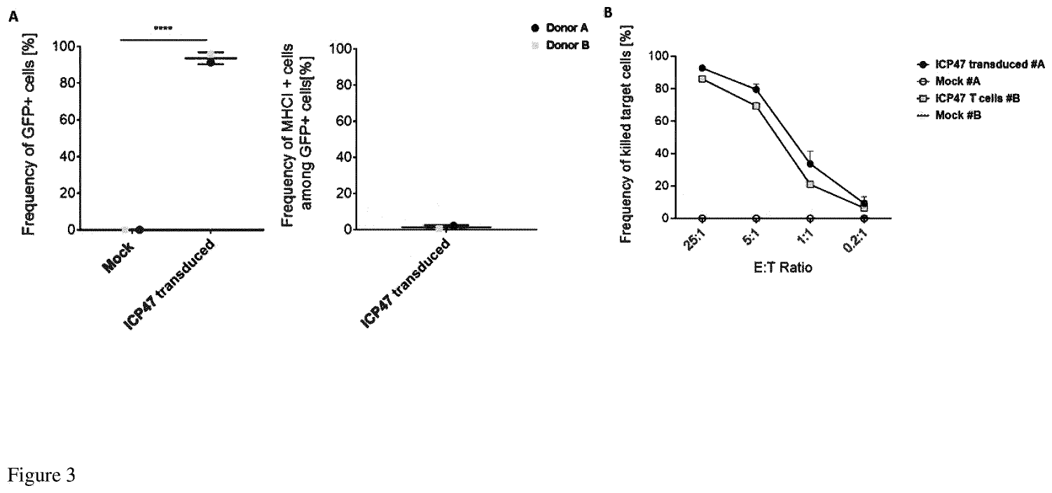

MHC I expression was frequently monitored using the MHC I-specific antibody HLA-ABC Antibody, anti-human, REAfinity™ (FIG. 2 and FIG. 3A).

### 1.3 Co-Culture of TCP47-Engineered T Cells and Autologous NK Cells

On day 8 after activation, 2E4 T cells were co-cultured with autologous NK cells, respectively, at different E:T ratios for 18-24 h. Subsequently, killing was analyzed using flow cytometry (FIG. 3B).

### Example 2. Genetic Modification of T Cells with Additional Immunoevasins

### 2.1 Assessing the Potential of K3 and US6 to Eliminate T Cells

Both K3 (UniProt ID: P90495) as well as US6 (UniProt ID: P14334) encoding sequences were subcloned into a lentiviral vector under the control of a PGK promoter, respectively. Lentiviral particles were produced as described under 1.1. Subsequently, T cells as well as NK cells were isolated and cultured as described under 1.2. T cells were transduced 24 h after activation with fresh or frozen, carefully resuspended VSV-G pseudotyped lentiviral particles encoding either K3 or US6, respectively. ICP47 encoding lentiviral particles were used as control.

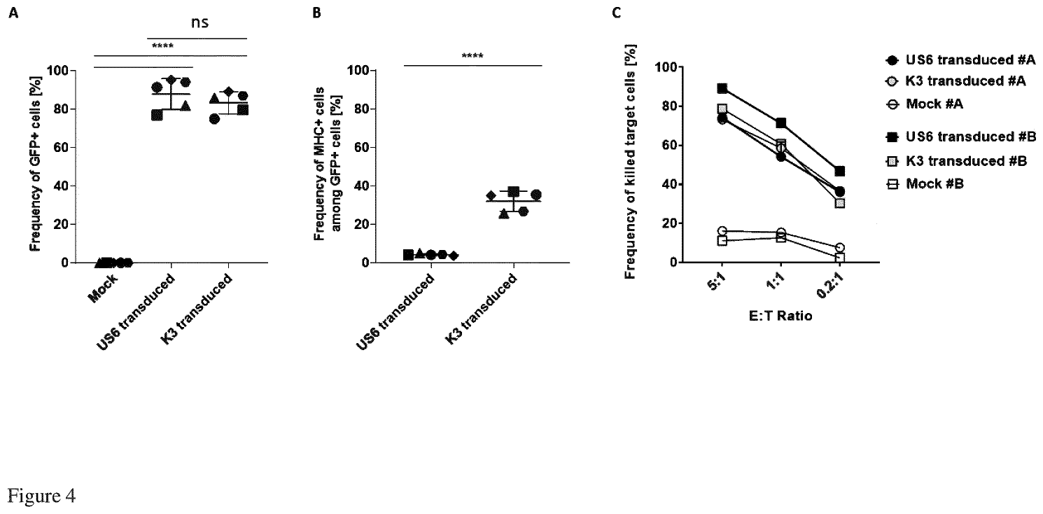

MHC I expression among GFP-positive T cells on day 6 was monitored using the MHC I-specific antibody HLA-ABC Antibody, anti-human, REAfinity™ (FIGS. 4A and 4B).

On day 8, either K3-, US6- or ICP47-modified T cells were co-cultured with autologous NK cells at different E:T ratios for 18-24 h. Subsequently, killing was analyzed using flow cytometry (FIG. 4C).

### REFERENCES

1. Lilley, B. N. and Ploegh, H. L., Viral modulation of antigen presentation: manipulation of cellular targets in the ER and beyond. Immunol Rev, 2005. 207: p. 126-44.
2. Zhou, X., Naik, S., Dakhova, O., et al., Serial Activation of the Inducible Caspase 9 Safety Switch After Human Stem Cell Transplantation. Mol Ther, 2016. 24(4): p. 823-31.
3. Budde, L. E., Berger, C., Lin, Y., et al., Combining a CD20 chimeric antigen receptor and an inducible caspase 9 suicide switch to improve the efficacy and safety of T cell adoptive immunotherapy for lymphoma. PLoS One, 2013. 8(12): p. e82742.
4. Philip, B., Kokalaki, E., Mekkaoui, L., et al., A highly compact epitope-based marker/suicide gene for easier and safer T-cell therapy. Blood, 2014. 124(8): p. 1277-87.
5. Yu, S., Yi, M., Qin, S., et al., Next generation chimeric antigen receptor T cells: safety strategies to overcome toxicity. 2019. 18(1): p. 125.
6. Kagoya, Y., Guo, T., Yeung, B., et al., Genetic Ablation of HLA Class I, Class II, and the T-cell Receptor Enables Allogeneic T Cells to Be Used for Adoptive T-cell Therapy. 2020. 8(7): p. 926-936.
7. Torikai, H., Reik, A., Soldner, F., et al., Toward eliminating HLA class I expression to generate universal cells from allogeneic donors. Blood, 2013. 122(8): p. 1341-9.
8. Ruella, M., Xu, J., Barrett, D. M., et al., Induction of resistance to chimeric antigen receptor T cell therapy by transduction of a single leukemic B cell. 2018. 24(10): p. 1499-1503.
9. Jones, T. R., Wiertz, E. J., Sun, L., et al., Human cytomegalovirus US3 impairs transport and maturation of major histocompatibility complex class I heavy chains. Proc Natl Acad Sci USA, 1996. 93(21): p. 11327-33.
10. Hewitt, E. W., Gupta, S. S., and Lehner, P. J., The human cytomegalovirus gene product US6 inhibits ATP binding by TAP. Embo j, 2001. 20(3): p. 387-96.
11. Wiertz, E. J., Jones, T. R., Sun, L., et al., The human cytomegalovirus US11 gene product dislocates MHC class I heavy chains from the endoplasmic reticulum to the cytosol. Cell, 1996. 84(5): p. 769-79.
12. Bartee, E., Mansouri, M., Hovey Nerenberg, B. T., et al., Downregulation of major histocompatibility complex class I by human ubiquitin ligases related to viral immune evasion proteins. J Virol, 2004. 78(3): p. 1109-20.
13. Lorenzo, M. E., Jung, J. U., and Ploegh, H. L., Kaposi's sarcoma-associated herpesvirus K3 utilizes the ubiquitin-proteasome system in routing class major histocompatibility complexes to late endocytic compartments. J Virol, 2002. 76(11): p. 5522-31.

1. A composition comprising
A) immune cells comprising
a) an inducible gene expression system comprising
I) a first nucleic acid comprising a drug-inducible promoter operably linked to a second nucleic acid
II) said second nucleic acid encoding a polypeptide or a non-coding RNA (ncRNA) which decreases cell surface expression level of major histocompatibility complex (MHC) class I relative to cell surface expression level of MHC class I of an immune cell that does not express said polypeptide or ncRNA
b) a third nucleic acid encoding a chimeric antigen receptor (CAR) or T cell receptor (TCR),
B) a drug that induces said drug-inducible promoter.

2. The composition according to claim 1, wherein said immune cells are T cells.

3. The composition according to claim 1, wherein said polypeptide is a viral protein which decreases cell surface expression level of major histocompatibility complex (MHC) class I relative to cell surface expression level of MHC class I of an immune cell that does not express the viral protein.

4. The composition according to claim 3, wherein said viral protein is from a virus selected from the group consisting of human cytomegalovirus (hCMV), murine cytomegalovirus (mCMV), rhesus cytomegalovirus (RhCMV), Epstein Barr virus (EBV), herpes simplex virus (HSV), bovine herpes virus-1 (BoHV-1), adenovirus (AV), coxpox virus (CV), Kaposi's sarcoma-associated herpesvirus (KSHV), mouse herpesvirus 68 (MHV68) or human immunodeficiency virus (HIV) or wherein the viral protein is selected from the group consisting of EBNA1 (EBV-derived), E3-19K (AV-derived), CPXV203 (CV-derived), mK3 (MHV68-derived), gp48 (mCMV-derived), K3 (KSHV-derived), K5 (KSHV-derived) or Nef (HV-derived).

5. The composition according to claim 4, wherein the viral protein is from hCMV and is selected from the group consisting of US2, US3, US6, US10, US11, UL40, UL82, UL83, miR-376a and miR-US4-1 and UL18.

6. The composition according to claim 4, wherein the viral protein inhibits transporter associated with antigen processing (TAP).

7. The composition according to claim 6, wherein the viral protein is selected from the group consisting of US6, ICP47, UL49.5 and BNLF2a.

8. The composition according to claim 1, wherein said inducible gene expression system further comprises a nucleic acid encoding a synthetic transcription factor for said drug-inducible promoter, wherein when a drug is administered to said immune cell, the gene expression system is induced and said polypeptide or said ncRNA is expressed.

9. The composition according to claim 8, wherein said synthetic transcription factor comprises a DNA binding domain and drug-binding domain and an activation domain, wherein said synthetic transcription factor is activated by binding to said drug.

10. The composition according to claim 1, wherein said composition comprises additionally,
C) autologous NK cells,
Wherein 90% of said immune cells are eliminated within 18 hours by said NK cells, when said polypeptide or said ncRNA is expressed in said immune cell and MHC I is reduced on the surface of said immune cell.

11. A composition according to claim 1 for use in immunotherapy for reducing or preventing side-effects associated with an immune cell therapy in a subject, wherein said immune cells are autologous cells.

12. An in-vitro method for generating engineered immune cells, the method comprising modifying immune cells by introduction into said immune cells
a) an inducible gene expression system comprising
I) a first nucleic acid comprising a drug-inducible promoter operably linked to a second nucleic acid
II) said second nucleic acid encoding a polypeptide or ncRNA which decreases cell surface expression level of major histocompatibility complex (MHC) class I relative to cell surface expression level of MHC class I of an immune cell that does not express the polypeptide or ncRNA
b) a third nucleic acid encoding a chimeric antigen receptor (CAR) or TCR,
wherein when a drug that induces said drug-inducible promoter is administered to said immune cells, the gene expression system is induced and said polypeptide or ncRNA is expressed.

13. The method according to claim 12, wherein said polypeptide is a viral protein which decreases cell surface expression level of major histocompatibility complex (MHC) class I relative to cell surface expression level of MHC class I of an immune cell that does not express the viral protein.

14. A combination of pharmaceutical compositions comprising
A) a composition of immune cells comprising
a) an inducible gene expression system comprising
I) a first nucleic acid comprising a drug-inducible promoter operably linked to a second nucleic acid
II) said second nucleic acid encoding a polypeptide or ncRNA which decreases cell surface expression level of major histocompatibility complex (MHC) class I relative to cell surface expression level of MHC class I of an immune cell that does not express the polypeptide or ncRNA
b) a third nucleic acid encoding a chimeric antigen receptor (CAR) or TCR,
and optional a pharmaceutical acceptable carrier, and
B) a composition of a drug that induces said drug-inducible promoter,
and optional pharmaceutical acceptable carrier.

15. The combination of pharmaceutical compositions according to claim 14, wherein said polypeptide is a viral protein which decreases cell surface expression level of major histocompatibility complex (MHC) class I relative to cell surface expression level of MHC class I of an immune cell that does not express the viral protein.
